(19) United States
(12) Patent Application Publication
(10) Pub. No.: US 2009/0117571 A1
(43) Pub. Date: May 7, 2009

(54) IMPEDANCE SPECTROSCOPY OF BIOMOLECULES USING FUNCTIONALIZED NANOPARTICLES

(75) Inventors: Rajendra Solanki, Portland, OR (US); Jeffrey S. King, Portland, OR (US); Kanwar Vikas Singh, Portland, OR (US)

Correspondence Address: MARGER JOHNSON & MCCOLLOM, P.C., 210 SW MORRISON STREET, SUITE 400, PORTLAND, OR, 97204, US

(73) Assignee: State of Oregon by and through the State Board of Higher Education on behalf of Portland State Univ., Portland, OR (US)

(21) Appl. No.: 12/192,822

(22) Filed: Aug. 15, 2008

Related U.S. Application Data
(60) Provisional application No. 60/956,108, filed on Aug. 15, 2007.

(51) Int. Cl. C12Q 1/68; C12M 1/34

(52) U.S. Cl. 435/6; 435/287.2

(57) ABSTRACT

A biosensor system includes a functionalized interdigitated electrode, functionalized nanoparticles, a current/voltage signal generator, and a circuit analyzer. The interdigitated electrode can be functionalized by coating an exposed surface with first biomolecular probes. The nanoparticles are functionalized by coating an outer surface with second biomolecular probes. A signal generator provides a signal (e.g., an alternating current or voltage) having a selected range of frequencies. A circuit analyzer analyzes electrical parameters of the circuit as the signal is applied. Sensitivity is increased by the presence of functionalized nanoparticles in the system. An analytic method includes measuring changes in electrical parameters of the circuit over the range of frequencies. Using these measurements, the biosensor system can determine whether a target biomolecule is bound. The biosensor system can also identify a biomolecule by comparing the detected signal or “electro-fingerprint” with a reference set of signals over the frequency range.

### CROSS-REFERENCE TO RELATED APPLICATION

This application claims benefit of U.S. Provisional Patent Application Ser. No. 60/956,108, filed Aug. 15, 2007, titled Impedance Spectroscopy of Biomolecules Using Functionalized Nanoparticle, which we incorporate in its entirety.

### BACKGROUND OF THE INVENTION

The present invention is related to the field of bioelectrical analyzers, and more specifically to bioelectrical analyzers and methods of impedance spectroscopy.

Detection of antigens associated with various diseases is critical for proper medical diagnoses. Various multi-step techniques currently exist for clinical detection of immunological conditions, including enzyme-linked immunosorbent assay (ELISA) and immunoradiometric assay (IRMA). However, the very multi-step nature of these techniques tends to make them prone to error, as well as time-consuming and expensive. Thus, there is considerable effort directed towards development of microsensors, in particular immunosensors that can allow quick and precise detection of biomolecules.

Biosensors are analytical devices that combine biologically sensitive elements with optical, chemical, or mechanical transducers for selectively and quantitatively detecting biomolecules. Biosensor technology has mostly focused on potentiometric, piezoelectric and capacitive systems. However, each of these systems has its downfalls. Potentiometric measurements tend to be non-specific, while piezoelectric systems suffer from instabilities and problems with calibration. Thus, while a great need for electronic-based biosensors for diagnostic assay applications exists, the current technology has been limited by low sensitivity and specificity.

Impedance spectroscopy can be used with biosensors to detect an “electro-fingerprint,” a unique pattern of electrical changes as a function of the electrical frequency. Impedance spectroscopy uses an electrical probe pulse over a specific frequency range to measure electrical parameters producing the “electro-fingerprint,” as taught in U.S. Pat. No. 7,214,528. Other methods and devices for applying an electrical field to a biosensor are described in U.S. Pat. Nos. 6,264,825; 6,602,400; and 6,716,620. A review of impedance spectroscopy in biomolecular screening is described by K'Owino et. al., Impedance spectroscopy: a powerful tool for rapid biomolecular screening and cell culture monitoring, Electroanalysis 17, 2101-2113 (2005). Further, an electrochemical immunosensor constructed by self-assemble technique and studied by impedance spectroscopy is described by Zhu et. al., An electrochemical immunosensor for assays of c-reactive protein, Anal. Lett. 36, 1547-1556 (2003).

The biomolecules detected in biosensors are generally basic functional units of biological systems such as enzymes, nucleic acids, antibodies, antigens, and cytokines, all of which are nanoscale in size. For example, the size of typical proteins is on the order of 2-50 nm, with proteins such as antibodies being about 15 nm in size. To appropriately characterize these nanoscale structures, a label of similar dimension is relevant. Whitesides, G. M., The “right” size in nanobiotechnology, Nature Biotech. 21, 1161-1165 (2003).

Nanoparticles are one type of label of similar dimension. Nanoparticles are zeroth order quantum structures, also referred to as quantum dots (QDs). They are also considered as artificial atoms because their electronic energy levels can be precisely chosen through variation of their diameters. The development of colloidal nanoparticles in solution has led to the application of nanoparticles for a wide range of medical diagnostics, generally falling into one of three categories: optical, magnetic, and electrical. Optical detection remains the most widely used mechanism for detecting biological binding events and for imaging in biological systems. Magnetic nanocrystals are also widely employed in artificial biological detection and separation systems, serving important roles as magnetic resonance contrast enhancement agents and the basis for a wide range of magnetophoresis experiments. Electrical detection of biomolecular interactions between polypeptides based on the conductance variation of a nanometer size-gap (typically less than 100 nm) between two planar electrodes has been described by Olivier et. al., Combined nanogap particles nanosensor for electrical detection of biomolecular interactions between polypeptides, Appl. Phys. Lett. 84, 1213-1215 (2004).

Although optical techniques continue to evolve, electrical detection remains extremely desirable. This is because the advances in microelectronics can be utilized to miniaturize and integrate these sensors into larger electronic systems that will be more robust and less expensive than optical-based systems.

Accordingly, a need remains for an improved method and apparatus for detecting biomolecules of interest in a sample analyte with increased specificity and sensitivity.

### SUMMARY OF THE INVENTION

The present invention includes a method of detecting and analyzing biomolecules in a sample analyte. The method includes providing a plurality of electrodes with an exposed surface. The electrodes may be formed in an interdigitated relationship. In one embodiment, the electrodes may further be functionalized by coating the exposed surface with a plurality of first biomolecular probes. The first biomolecular probes may include an antigen, an antibody, a secondary antibody, an isotype, an enzyme, a nucleic acid, a cytokine, a peptide, or any combination thereof.

The method further includes functionalizing a plurality of metallic nanoparticles by coating an outer surface of the nanoparticles with a plurality of second biomolecular probes. The second biomolecular probes may include an antigen, an antibody, a secondary antibody, an isotype, an enzyme, a nucleic acid, a cytokine, a peptide, or any combination thereof. The functionalized nanoparticles may be gold nanoparticles, silver nanoparticles, iron nanoparticles, iron oxide nanoparticles, platinum nanoparticles, palladium nanoparticles, or any combination thereof.

The method also includes applying the plurality of functionalized metallic nanoparticles and a sample analyte to the plurality of electrodes. The functionalized metallic nanoparticles may be applied to the electrodes separately from the sample analyte. The functionalized metallic nanoparticles may alternatively be first combined with the sample analyte to form a mixture that is then applied to the electrodes.

The method also includes using impedance spectroscopy to detect a sample signal profile for a group of sample electrical parameters across a selected frequency range. The selected frequency range may include 25 Hz to 50 kHz. The parameters may include impedance, capacitance, dissipation factor, phase, or any combination thereof. The method may further include comparing the sample signal profile with a reference sample signal profile to detect a match across the selected frequency range.

The present invention also includes a biosensor system for detecting or identifying biomolecules in a sample analyte. The biosensor system includes a substrate, and an electrode formed on the substrate. The electrode includes one or more pairs of opposed fingers, and each finger has an exposed upper surface and exposed side walls. The electrode may be functionalized with a plurality of first biomolecular probes. The first biomolecular probes may include an antigen, an antibody, a secondary antibody, an isotype, an enzyme, a nucleic acid, a cytokine, a peptide, or any combination thereof.

The biosensor system also includes a stimulator electrically coupled to the electrode and structured to provide a plurality of input frequencies over a selected frequency range. The selected frequency range may include 25 Hz to 50 kHz.

The biosensor system also includes a detector operative to detect a signal of a sample analyte over the selected frequency range and generate a sample signal profile for a group of sample electrical parameters. The parameters may include impedance, capacitance, dissipation factor, phase, or any combination thereof.

The biosensor system also includes means for comparing the sample signal profile with a reference signal profile to detect a substantial match across the selected frequency range.

The biosensor system also includes a plurality of functionalized nanoparticles. The nanoparticles may be functionalized with a plurality of second biomolecular probes. The second biomolecular probes may include an antigen, an antibody, a secondary antibody, an isotype, an enzyme, a nucleic acid, a cytokine, a peptide, or any combination thereof. The functionalized nanoparticles may be gold nanoparticles, silver nanoparticles, iron nanoparticles, iron oxide nanoparticles, platinum nanoparticles, palladium nanoparticles, or any combination thereof.

The present invention also includes a method of detecting and analyzing biomolecules in a reference sample analyte. The method includes providing a plurality of electrodes, each electrode having an exposed surface. The electrodes may be functionalized by coating the exposed surface with a plurality of first biomolecular probes. The first biomolecular probes may include an antigen, an antibody, a secondary antibody, an isotype, an enzyme, a nucleic acid, a cytokine, a peptide, or any combination thereof.

The method further includes functionalizing a plurality of metallic nanoparticles. The metallic nanoparticles may be functionalized by coating an outer surface with a plurality of second biomolecular probes. The second biomolecular probes may include an antigen, an antibody, a secondary antibody, an isotype, an enzyme, a nucleic acid, a cytokine, a peptide, or any combination thereof.

The method also includes applying the plurality of functionalized metallic nanoparticles and the reference sample analyte to the plurality of electrodes. The functionalized metallic nanoparticles may be applied to the electrodes separately from the reference sample analyte. The functionalized metallic nanoparticles may alternatively be first combined with the reference sample analyte to form a mixture that is then applied to the electrodes. Impedance spectroscopy is used to detect a reference sample signal profile for a group of sample electrical parameters across a selected frequency range.

The method may further include storing the reference sample signal profile in a database for future comparison to a detected sample signal profile of a sample analyte. The method may also further include subtracting a signal of a buffer solution from a reference sample signal to obtain the reference sample signal profile.

The foregoing and other features, objects and advantages of the various aspects of the invention will become more readily apparent from the following detailed description of preferred and alternative embodiments, and examples with reference to the accompanying drawings.

### BRIEF DESCRIPTION OF THE DRAWINGS

FIG. 1 is a top view of an interdigitated electrode usable in an embodiment of the present invention.

FIG. 2 is an enlarged cross-sectional view of a portion of the interdigitated electrode taken along line 2-2′ of FIG. 1.

FIG. 3A is a depiction of the interdigitated electrode of FIG. 2A after it has been functionalized with a plurality of first biomolecular probes.

FIG. 3B is a depiction of the interdigitated electrode of FIG. 3A after a mixture of sample analyte and functionalized nanoparticles has been added to the interdigitated electrode.

FIG. 4A is a depiction of the interdigitated electrode of FIG. 3A after a sample analyte has been added to the electrode.

FIG. 4B is a depiction of the interdigitated electrode of FIG. 4A after functionalized nanoparticles have been added to the interdigitated electrode.

FIG. 5A is a cross-sectional view of an embodiment of a biosensor system wherein two groupings of interdigitated electrodes are arranged linearly on a substrate such that a single sample may be flowed over each individual interdigitated electrode.

FIG. 5B is an enlarged top view of one of the groupings of interdigitated electrodes seen in FIG. 5A.

FIG. 6 is a block diagram of an embodiment of a device for electrically detecting a molecule-molecule interaction enhanced by the presence of functionalized nanoparticles in the system.

FIG. 7 is a diagram of a preferred method for detecting and analyzing a group of sample electrical parameters in a biosensor system utilizing functionalized nanoparticles.

FIGS. 8-12 are graphs presenting experimental results generated using a bioanalyzer system and method as described herein.

### DETAILED DESCRIPTION

The present disclosure describes a platform which combines microelectronic sensors and novel biological materials and methods with nanoparticles to develop immunosensors with improved sensitivity. The system uses impedance spectroscopy to detect distinct molecular interactions for the rapid, direct detection of single molecular species, or for simultaneously testing for multiple agents. This disclosure is an improvement of a methodology that identifies biological or non-biological molecules by their response to an electrical probe pulse over a specific frequency range, as disclosed in U.S. Pat. No. 7,214,528.

General Configuration of the System

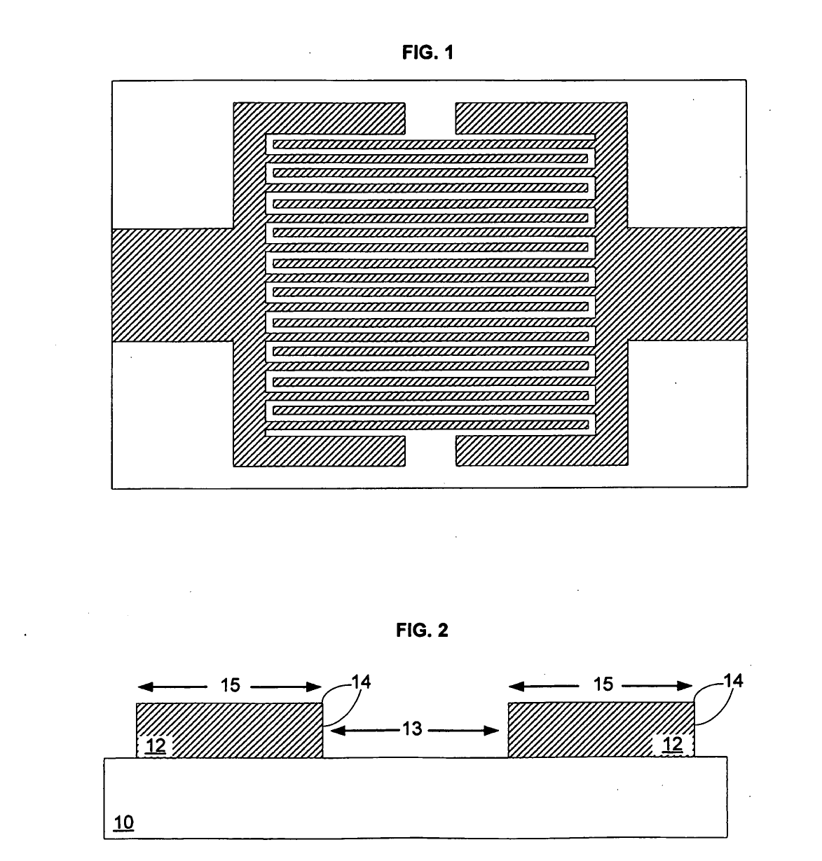

FIG. 1 illustrates a top view of a preferred embodiment of an electrode 11. The electrode 11 is an interdigitated electrode (IDE) and has interdigitated fingers 12 that are formed using standard processes used in fabrication of microelectronic devices. First, a layer of silicon dioxide (not shown) is deposited or grown on the surface of a substrate 10. The substrate 10 may be composed of silicon, glass, or plastic. Then, a metal film is deposited on the silicon dioxide layer. The metal film may be composed of any conductive metal such as chromium, gold, iron, platinum, or palladium. Subsequently, photolithography is used to pattern the electrode 11 and its interdigitated fingers 12, followed by plasma etching of the metal film to produce the electrodes 11. The IDE configuration of the electrode 11 maximizes the electrical interaction between a sensor of the system and a sample analyte.

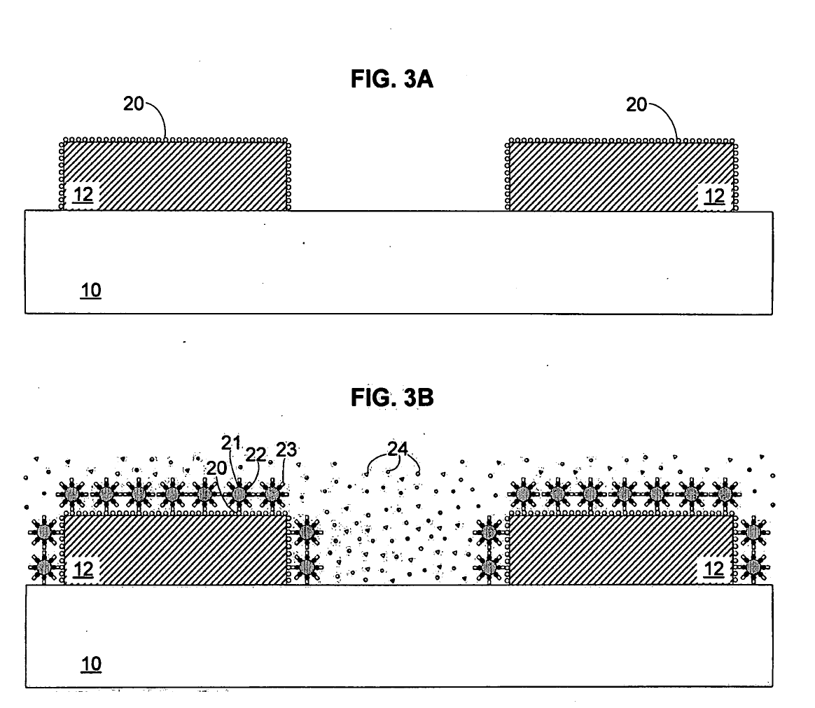

FIG. 2 illustrates an enlarged cross-sectional view of a pair of interdigitated fingers 12, the cross-section taken through line 2-2′ of FIG. 1. The electrode 11 and its interdigitated fingers 12 are formed such that they are substantially raised above the substrate 10. Thus, the electrode 11 and its interdigitated fingers 12 have an exposed surface 14 that includes both a top surface of, and side walls of, the electrode 11 and fingers 12. Thus, the electrode 11 and its interdigitated fingers 12 are raised, having a particular thickness (e.g., 250 nm). This raised configuration is important, as experiments have shown that planar configurations are significantly inferior. The vertical wells created by the raised configuration allow for binding proteins to partially bridge the gap 13 between adjacent fingers 12 and thus significantly alter electrical properties of the system. This bridging between opposing side walls of the electrodes significantly increases the specificity of the system, as different proteins will bridge different proportions of the gap dependent on the molecular size, configuration, and conductivity.

Both the gap 13 between interdigitated fingers 12, and the width 15 of the interdigitated fingers 12, can be varied. However, an optimal configuration for the fingers 12 was determined to be a gap 13 of 0.5 μm, and a width 15 of 0.5 μm. The optimal configuration was determined by a two-step process. First, a background impedance spectrograph with only a MOPS buffer solution present on the chip surface was collected. An antibody (either anti-DNP or anti-gp41) was then added and another impedance spectrograph was acquired for comparison. The first spectrograph was subtracted from the second and the resultant background-subtracted spectrograph showed the effect of the antibody on the electrical characteristics of the chip.

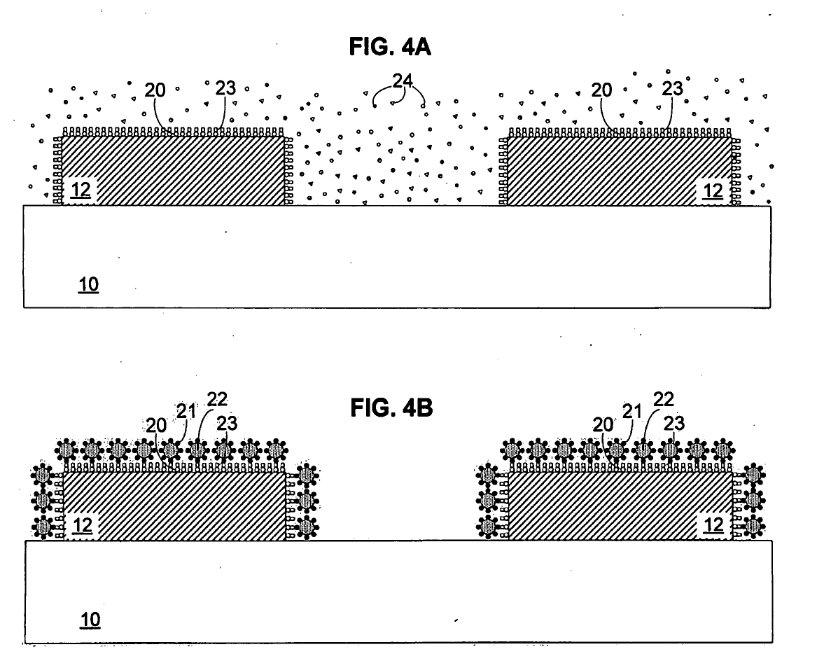

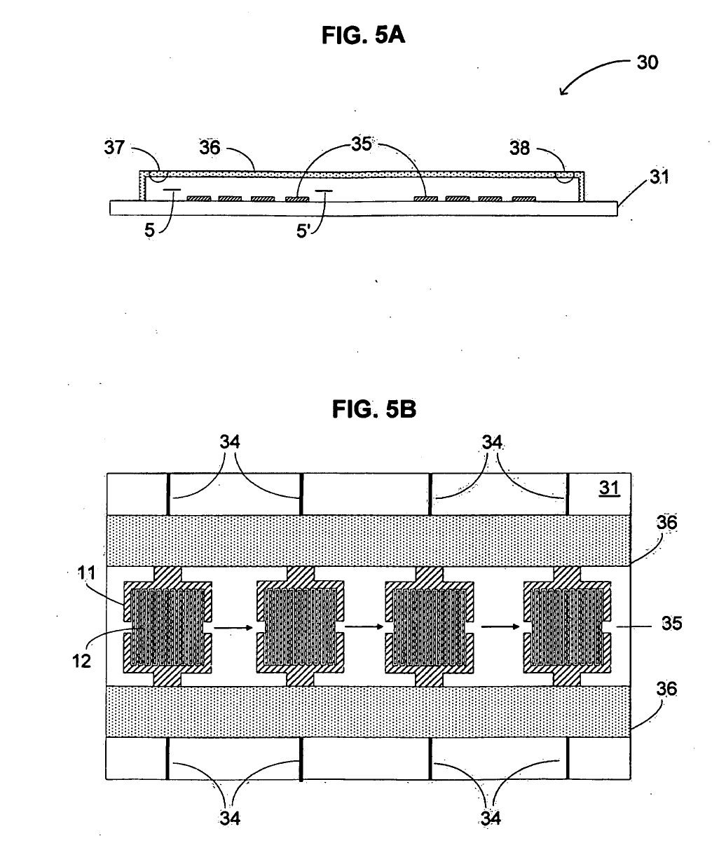

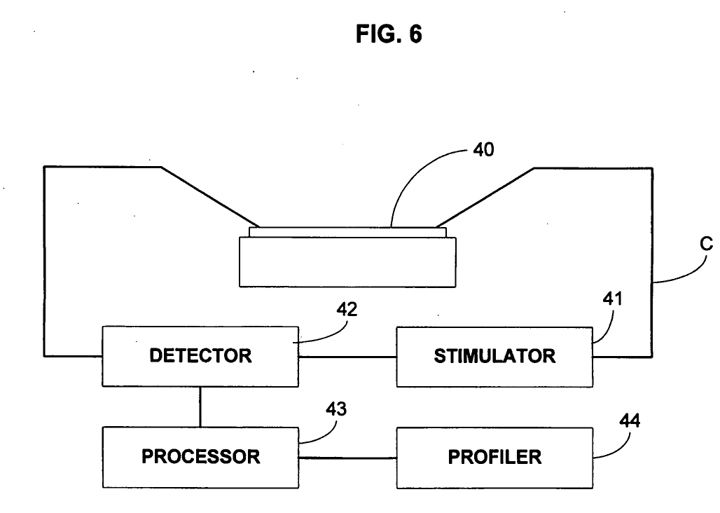

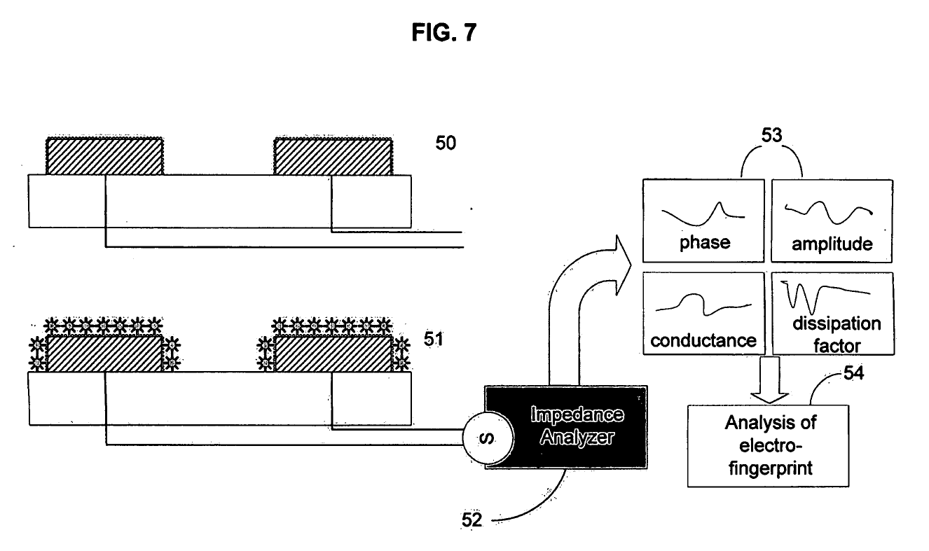

The spectrographs from each configuration were compared to determine the configuration that most effectively displayed the signal produced by the addition of antibody. After considering the data, the ease of data collection, and the physical size of the different configurations, it was determined that the raised 0.5 um gap 13 configuration was optimal, with no significant differences observed between electrode widths. This is in agreement with Anasoft computer simulations that suggest that electric field strength is highest at the corners of the fingers 12 and in the gap 13 between the fingers 12. The electric field strength falls off rapidly moving away from the corners of the fingers 12, resulting in low field strength at the top of the fingers 12. However, a person of skill in the art will recognize that any width of gap 13 which is on an order of magnitude greater than a length and a width of a nanoparticle 22 (FIGS. 3B, 4B) used in the system may suffice. Nanoparticles may typically range in size from 10-100 nm.

FIGS. 3A-3B illustrate a preferred embodiment for a biosensor system incorporating functionalized nanoparticles. FIG. 3A illustrates the pair of interdigitated fingers 12 of FIG. 2 after the electrode 11 has been functionalized with a plurality of first biomolecular probes 20. In general, functionalizing occurs when an object, like electrode 11, has one or more functional groups adhered to its surface. Functional groups are generally small chemical groups such as an amine (—NH2), a carboxyl (—COOH), or a thiol (—SH) which act as anchors for the attachment of biomolecules, such as first biomolecular probe 20, on a given surface, such as electrode 11. Once the electrode 11 is functionalized, first biomolecular probes 21 may be chemically attached to its exposed surface using similar functional groups present on the first biomolecular probes 21.

Because the biomolecules that adhere to the functional groups are capable of interacting with other biomolecules introduced to a system, the object to which the functional groups and biomolecules are adhered becomes functional, or “functionalized.” Thus, as shown in FIG. 3A, when an exposed surface of a pair of interdigitated fingers 12 of an electrode 11 are coated in a plurality of first biomolecular probes 20 such as an antigen through the use of functional groups, the interdigitated fingers gain functionality and can bind other biomolecules such as biomolecular target 23 (FIG. 3B).

Although only one pair of interdigitated fingers 12 are depicted as being functionalized in FIG. 3A, it should be apparent that all of the interdigitated fingers 12 of a given electrode 11 (see FIG. 1) are simultaneously functionalized. Functionalizing a metal surface can be achieved either through passive absorption (such as inherent chemical properties of a chromium metallic electrode) or through active absorption (such as first coating the electrode surface with a polymer, a silane, or thiol-based self assembled monolayers providing chemical functionalities (or functional groups) to facilitate binding of biomolecular probes). Further, the first biomolecular probes 20 can be an antigen as described above, but they can also be any protein which specifically binds a biomolecular target 23 (FIG. 3B), such as an antibody, an enzyme, a nucleic acid, or a cytokine.

FIG. 3B illustrates the pair of interdigitated fingers 12 of FIG. 3A after a mixture of sample analyte and functionalized nanoparticles 22 is added to the electrode 11. Similar to the metallic electrode 11, nanoparticles 22 can acquire functionality through the adherence of functional groups to their outer surface. Adherence of functional groups is achieved through chemical alteration of the nanoparticle 22 surface such that it is able to chemically attach functional groups to which a second biomolecular probe 21 can bind. Functionalizing nanoparticles can be achieved through the same passive and active absorption means described above with respect to the electrodes. Alternatively, the nanoparticles 22 can first be coated in a surfactant (eg PEGylated-lipids) (not shown) in the manner of a microemulsion such that micellar nanoparticles 22 are formed. Thus, the hydrophilic surface of the nanoparticles 22 can be coated with functional groups, which can be used for binding with second biomolecular probes 21. Such micellar nanoparticles have an advantage in that they do not adhere to each other, and are suitable for biological and biomedical applications.

The nanoparticles 22 are preferably metallic nanoparticles, and can potentially comprise gold nanoparticles, silver nanoparticles, iron nanoparticles, iron oxide nanoparticles, platinum nanoparticles, palladium nanoparticles, or a combination thereof. Besides nanoparticles, one can also use functionalized nanowires or nanotubes.

The metallic nanoparticles 22 are thus functionalized by adhering a plurality of second biomolecular probes 21 to the outer surface of the metallic nanoparticles 22 through the use of functional groups. The second biomolecular probe 21 is specific for a biomolecular target 23 within a given sample analyte. For instance, the second biomolecular probe 21 may be a secondary antibody specific for the biomolecular target 23. However, the second biomolecular probe 21 can also be any protein which specifically binds a target biomolecule, such as an antibody, an enzyme, a nucleic acid, or a cytokine.

After the metallic nanoparticles 22 are functionalized, they can be mixed with a sample analyte, allowing for binding to occur between the second biomolecular probe 21 and the biomolecular target 23. This is achieved by mixing together the functionalized nanoparticles 22 and the sample analyte in a separate container, such as a test tube. In this manner, the biomolecular target 23 will bind to the functionalized nanoparticles 22, while non-specific biomolecules 24 in the sample analyte, which are unable to bind with the second biomolecular probe 21, will remain in solution. By mixing the functionalized nanoparticles 22 and sample analyte before adding either of them to the functionalized electrode 11, the process can be reduced to a single step, increasing overall efficiency of the process.

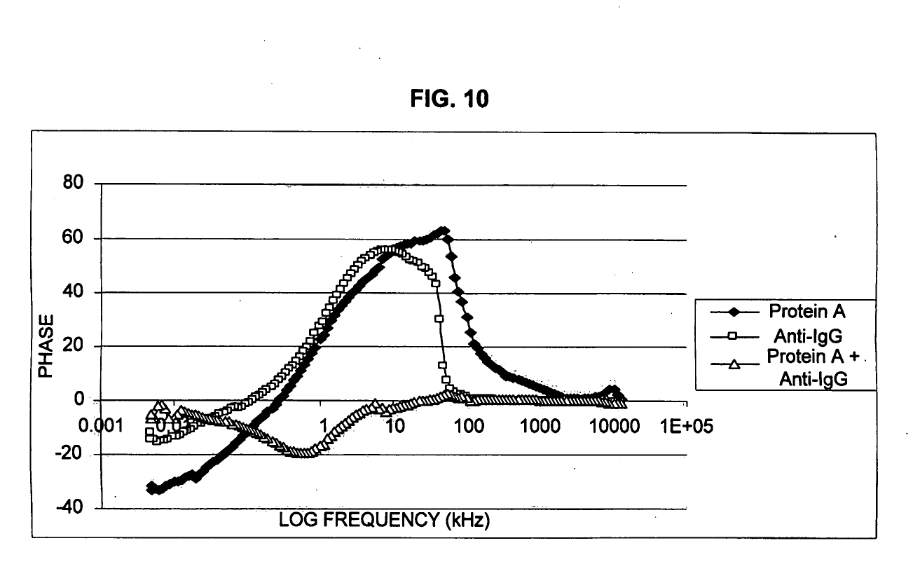

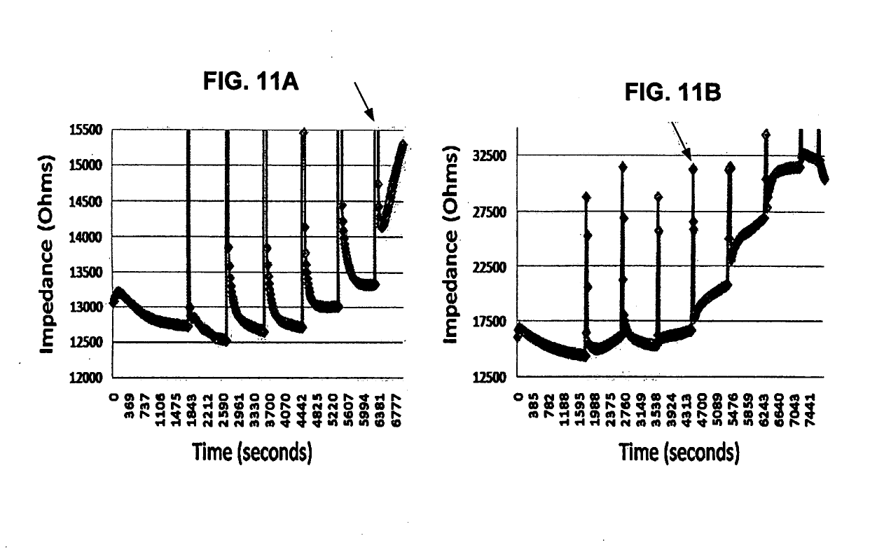

The resulting mixture may be added directly to the electrode 11. As the first biomolecular probes 20 on the electrode surface 14 are also specific for the biomolecular target 23, only the nanoparticle-biomolecular target conjugate will bind to the functionalized electrode 11. Non-specific biomolecules 24 will remain in solution and can thus be removed along with the solution from the system. Thus, when impedance spectroscopy over a range of frequencies is applied to the system, an electro-fingerprint specific for only the bound biomolecular target 23 can be obtained. The sensitivity of the system is enhanced by the presence of the functionalized metallic nanoparticles 22. The impedance spectroscopy will be discussed in more detail with regards to FIGS. 6-7 below.

While FIGS. 3A-3B illustrate an embodiment for a biosensor system wherein a sample analyte is added to the system, in another embodiment a reference sample analyte may be added to the system. For instance, the electrode 11 and its interdigitated fingers 12 may be functionalized with a particular first biomolecular probe 20, and the metallic nanoparticles 22 may be functionalized with a particular second biomolecular probe 21. Then, a reference sample analyte with a positive control (i.e., specific) biomolecular target 23 may be combined with the functionalized nanoparticles 22 to form a mixture of nanoparticle-biomolecular target which is then added to the system, and impedance spectroscopy may be used to gather a reference signal profile for the positive control. A second mixture of functionalized nanoparticles 22 and reference sample analyte with a negative control (i.e., non-specific biomolecules 24) may subsequently be added to the system, and impedance spectroscopy may be used to gather a reference signal profile for the negative control. These reference signal profiles may then be stored in a profiler 44 (FIG. 6) for future comparison to sample signal profiles.

Further, although FIG. 3A shows the electrode 11 and its interdigitated fingers 12 being functionalized with a biomolecular target 21, in an alternate embodiment this functionalization need not occur. In the alternate embodiment, only a plurality of metallic nanoparticles 22 are functionalized. The electrode 11 acts to electrically perturb sample target biomolecules 23 adhered to the second biomolecular probes 21 of the functionalized metallic nanoparticles 22 that are flown over its surface. This electrical perturbation results in a specific electrical fingerprint for the bound sample target biomolecule 23 when impedance spectroscopy signals are applied to the system.

FIGS. 4A-4B illustrate another embodiment for a biosensor system incorporating functionalized nanoparticles. FIG. 4A illustrates the pair of interdigitated fingers 12 of FIG. 3A after a sample analyte is added to the functionalized electrode 11. The sample analyte may contain both biomolecular targets 23 specific for the first biomolecular probes 20, and non-specific biomolecules 24 that do not bind with the first biomolecular probes 20. Thus, the biomolecular targets 23 will bind to the functionalized electrode 11, while the non-specific biomolecules 24 will remain in solution and can be removed along with the solution from the system.

FIG. 4B illustrates the functionalized electrode 11 of FIG. 4A after addition of functionalized nanoparticles 22 to the system. The non-specific biomolecules 24 are preferably washed away in an intermediate step (not shown). The nanoparticles 22 are functionalized with second biomolecular probes 21, as described above. The second biomolecular probe 21 may be any biomolecule specific for the biomolecular target 23, such as an antigen, an antibody, a secondary antibody, an enzyme, a nucleic acid, or a cytokine. The nanoparticles 22 are preferably metallic nanoparticles, and can potentially comprise gold nanoparticles, silver nanoparticles, iron nanoparticles, iron oxide nanoparticles, platinum nanoparticles, palladium nanoparticles, or a combination thereof. Functionalized nanowires or nanotubes may also be used.

The second biomolecular probe 21 binds to the biomolecular target 23. In this manner, the nanoparticles 22 become bound to the electrode 11 via a first biomolecular probe-biomolecular target-second biomolecular probe sandwich. When impedance spectroscopy signals over a range of frequencies are applied to the system, an electro-fingerprint specific for the bound biomolecular target 23 can be obtained. The sensitivity of the system is enhanced by the presence of the functionalized metallic nanoparticles 22. The impedance spectroscopy will be discussed in more detail with regards to FIGS. 6-7 below.

While FIGS. 4A-4B illustrate an embodiment for a biosensor system wherein a sample analyte is added to the system, in another embodiment a reference sample analyte may be added to the system. For instance, the electrode 11 and its interdigitated fingers 12 may be functionalized with a particular first biomolecular probe 20, and the metallic nanoparticles 22 may be functionalized with a particular second biomolecular probe 21. A reference sample analyte with a positive control (i.e., specific) biomolecular target 23 may be added to the system, allowing the positive control biomolecular target 23 to bind to the electrode 11. Functionalized nanoparticles 22 may be added to the system such that they bind to the bound positive control biomolecular target 23. Impedance spectroscopy may be used to gather a reference signal profile for the positive control. These steps may be repeated with a negative control, a nonspecific biomolecule 24, and impedance spectroscopy may be used to gather a reference signal profile for the negative control. These reference signal profiles may then be stored in a profiler 44 (FIG. 6) for future comparison to sample signal profiles.

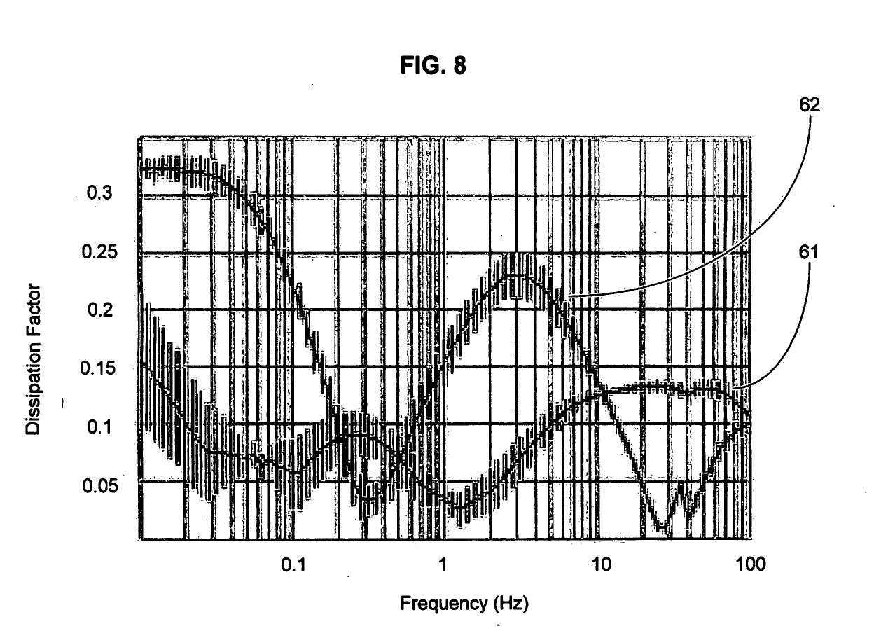

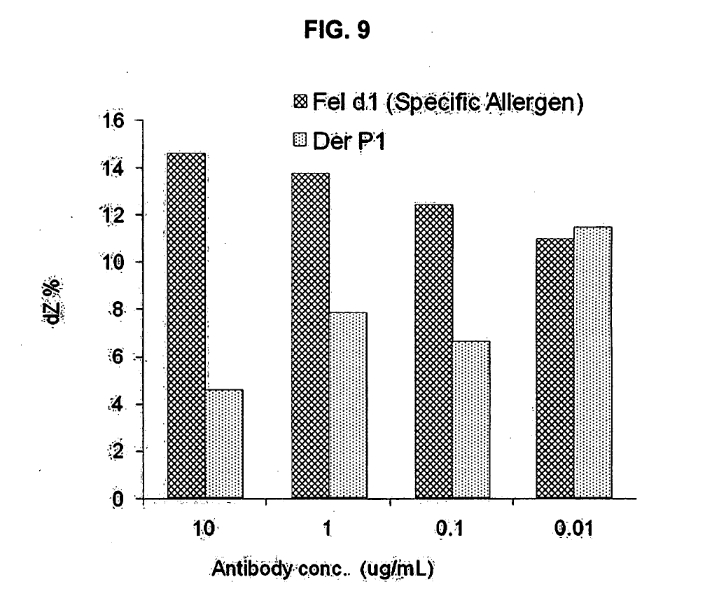

FIGS. 5A-5B illustrate an embodiment of a microelectrode fixture 30 for use in the described system is shown. FIG. 5A is a cross-sectional view of the microelectrode fixture 30. At least one row or grouping 35 of interdigitated electrodes is arranged linearly on a substrate 31. Each interdigitated electrode within a grouping 35 may be electrically connected to a contact pad (not shown), allowing groupings 35 of interdigitated electrodes to be directly connected to an electrical device comprising a detector 42 and a stimulator 41 (see FIG. 6). A representative electrical device is a Gamry Potentiostat, manufactured by Gamry Instruments of Warminster, Pa.

The microelectrode fixture 30 may further have a sample channel 36 which is disposed on the substrate 31 such that it covers and contains the groupings 35 of interdigitated electrodes. The sample channel 36 is structured such that a sample analyte may be introduced through a first port 37 at one end of the microelectrode fixture 30 and subsequently flowed over each interdigitated electrode within the groupings 35. The sample analyte may then be removed from the microelectrode fixture 30 through a second port 38 at the opposite end of the sample channel 36. Introducing and removing the sample analyte may be achieved, for example, by pipetting, or by attaching tubes (not shown) to either first port 37, second port 38, or both.

Each interdigitated electrode of the microelectrode fixture 30 may be functionalized with a different first biomolecular probe 20, or different functionalized nanoparticles 22 (see FIGS. 3A-3B and 4A-4B). Thus, multiple biomolecular targets 23 can be probed for simultaneously, using the same sample analyte. In this manner, multiplexing can be achieved, allowing for a greater number of biomolecular targets 23 to be probed for at a time, increasing efficiency of the system and decreasing the amount of sample analyte needed. Further, because the microelectrode fixture 30 is used in a system which also utilizes functionalized nanoparticles 22, higher sensitivity of each IDE within the microelectrode fixture 30 is achieved, and thus results can be obtained from smaller sample analyte volumes. A person of skill in the art will recognize that the system can be used with only a single line of groupings 35, as well as with microelectrode fixtures having a plurality of lines of groupings 35, i.e. a matrix. A person of skill in the art will also recognize that there may also be a plurality of individual IDEs within a grouping.

FIG. 5B is an enlarged top view of one grouping 35 of IDEs taken through line 5-5′ of FIG. 5A. Thus, the top of sample channel 36 has been cut away, allowing for an unobstructed view of the grouping 35. Each individual electrode 11 within each grouping 35 includes a plurality of interdigitated fingers 12. Each IDE also has its own connecting line 34. Further, arrows have been added to indicate a flow of a sample analyte moving to the right of the microelectrode fixture after it is added to the sample channel 36. However, a person of skill in the art will recognize that, depending on the current introduced to the system, the flow could alternatively move from the right to the left.

Detection Methodology

FIG. 6 illustrates an embodiment of a device for electrically detecting a molecule-molecule interaction enhanced by the presence of functionalized nanoparticles in the system. A circuit C is electrically coupled to one or more interdigitated electrodes 11 (FIG. 1), at least one of which may be functionalized with a plurality of first biomolecular probes 20 (FIGS. 3A, 4A). The metallic nanoparticles 22 are themselves functionalized with a plurality of second biomolecular probes 21, which are specific for a sample target biomolecule 23 (FIGS. 3B, 4B). Thus, the circuit C has imparted to it a biochemical quality by the functionalized electrodes 11 and functionalized nanoparticles 22. The portion of the system comprising the functionalized interdigitated electrodes 11 and functionalized nanoparticles 22 is represented by electrode module 40.

The circuit C can be further electrically coupled to a stimulator 41. The stimulator 41 is operative to provide an input alternating signal spanning a selected frequency range F₁-F₂. In a preferred embodiment, the frequency can have an F₁ value of 25 Hz, and an F₂ value of 50 kHz. Within this range, most actual detection may take place around the lower range nearing F₁, while the upper range nearing F₂ may be used more for quality control purposes. Thus, the addition of functionalized nanoparticles to the system increases the overall sensitivity of the system, allowing for detection at frequencies substantially lower than those of other biosensors.

The circuit C can also be further electrically coupled to a detector 42. The detector 42 is structured to detect and measure any one or more of a plurality of electrical parameters of the circuit C over the selected frequency range F₁-F₂. These electrical parameters include phase, amplitude, dissipation factor, and/or impedance, where the impedance parameters can also be represented by Nyquist plots. By analyzing the detected electrical parameter(s), the detector 42 can further generate a signal profile for a given biochemical circuit. This signal profile is an “electro-fingerprint” of the tested biochemical circuit, based on measurements of the electrical parameters at a plurality of points through the selected frequency range F₁-F₂. The components which impart a biochemical quality to the circuit C (the functionalized electrode 11 and the functionalized nanoparticles 22) all factor into the “electro-fingerprint” generated by the detector 42.

The detector 42 can be further electrically connected to means for analyzing the detected signal profile to determine what has been bound by the system. These means may include a computer or processor 43 configured to compare the detected signal profile to a reference signal profile stored in a profiler 44 (i.e., a memory) across the frequency range F₁-F₂. This comparison of profiles may be used to generate a match across the frequency range between the sample signal profile and a reference signal profile.

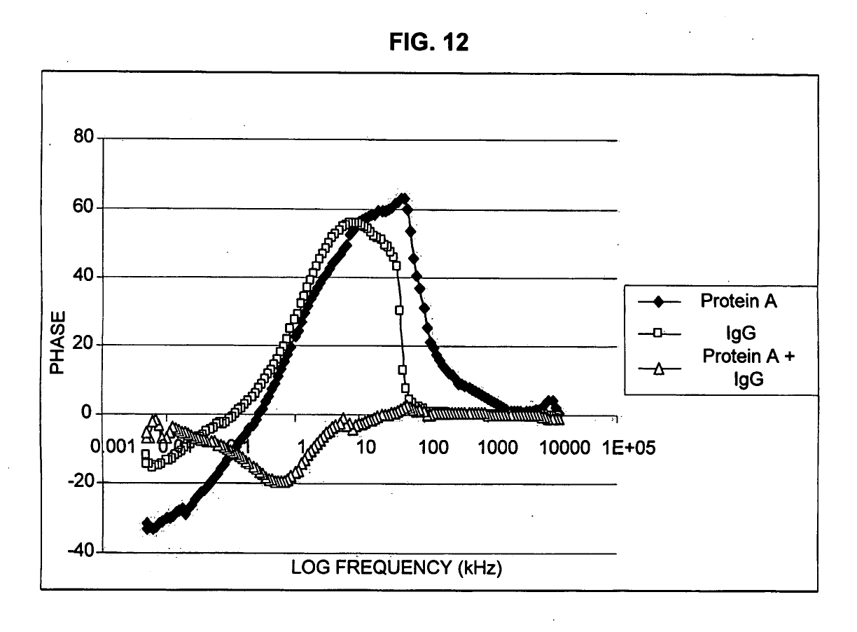

The profiler 44 may include a collection of spectra for a variety of known test samples to serve as a basis for comparison. The profiler 44 can also store a set of reference sample signal profiles. The reference sample signal profiles can be generated by applying a reference sample analyte to the biosensor system and generating an electro-fingerprint for the reference sample analyte. The reference sample analyte can be, for example, a positive control (i.e., a biomolecule capable of binding with the functionalized nanoparticle) in a low complexity buffer solution like a MOPS buffer solution. The reference sample analyte can also be a negative control (i.e., a non-binding biomolecule) in a low complexity buffer solution. A signal of the buffer solution can be subtracted from the reference sample signal to arrive at the reference sample signal profile. By comparing the detected signal profile with the database of reference sample signal profiles stored in the profiler 44, the computer or processor 43 can determine if binding occurred between the first biomolecular probe 20 and the biomolecular target 23. This comparison will be discussed in more detail below with reference to experimental data shown in FIGS. 8-12.

FIG. 7 summarizes a preferred method for detecting and analyzing a group of sample electrical parameters in a biosensor system which includes functionalized nanoparticles. First, the IDE is functionalized through the adherence of first biomolecular probes to its surface, at step 50.

Second, a sample analyte is combined with nanoparticles functionalized with second biomolecular probes to form a mixture, which is then added to the functionalized IDE, at step 51. The second biomolecular probes bind with sample target biomolecules to form target biomolecule-nanoparticle conjugates, while non-specific sample biomolecules remain in solution. When the mixture is added to the IDE, the sample target biomolecules bind to the first biomolecular probes on the electrode. Non-specific sample biomolecules do not bind, and thus remain in solution. These non-binding sample biomolecules are flowed out of the cell before measurements are taken. As already discussed, step 51 can alternatively be performed by adding the sample analyte to the IDE prior to addition of the functionalized nanoparticles (FIG. 4A-4B).

Third, an impedance analyzer is used as described in FIG. 6 to detect a signal profile over a given frequency range, shown at steps 52 and 53. The signal profile may comprise electrical parameters including phase, amplitude, conductance, and dissipation factor.

Finally, a processor 43 and a profiler 44 (FIG. 6) are used to compare the detected signal profile, or “electro-fingerprint,” to a set of reference signal profiles generated from reference sample analytes to determine what binding occurred in the system, shown at step 54. This comparison of profiles may be used to generate a match across the frequency range between the sample signal profile and a reference signal profile.

The biosensor system, and methods for detecting and analyzing using the biosensor system, will be further described hereafter with reference to experimental data shown in FIGS. 8-12.

Experimental Data

The biosensor system and method for using the same described herein are based on impedance spectroscopy and utilize basic principles of AC electronics to detect distinct molecular interactions. This procedure allows rapid, direct detection of single molecular species. The biosensor system and method can also be used to simultaneously test for multiple biomolecular agents.

The process utilizes a methodology that identifies biological or non-biological molecules by their response to an electrical probe pulse over a specific frequency range, and increases the sensitivity of that methodology by utilizing functionalized nanoparticles. This process produces an “electro-fingerprint” or unique pattern of electrical changes as a function of the electrical frequency. Electrical parameters including impedance/conductance, phase, capacitance, and dissipation factor are measured, resulting in a signal profile (response amplitude versus frequency) that is unique to the molecules between the sensor electrodes. The magnitude of the signals provides information on the concentration of target molecules.

The application of the electrical field produces polarization of the bound biomolecules and hence, changes in permittivity. The control variable for these measurements is the frequency of the alternating electric field. When an electric field is applied across a molecule, there is a tendency for the charges on the molecule to align with the applied field. In larger molecules, the electron cloud surrounding these molecules often redistributes, resulting in polarization of the molecule, i.e., an effective charge separation across the molecule. The ability of the charges to separate, and how fast this happens, depends on how strongly they are bound. Charges that are loosely bound can respond to the electric field at higher frequencies and vice versa. Hence, by looking at the response over a frequency range, one can examine specific traits of a given molecule. The capacitance scan also allows one to examine the dielectric response, which becomes dominant at lower frequencies. AC analysis can be used to determine the complex permittivity and admittance. A frequency sweep can show the resonance frequencies of dielectric loss or relaxation, i.e., when the dipole moment is strong enough to influence the permittivity.

To demonstrate the ability of the biosensor system to detect a specific antibody-antigen binding event, tests were run on the system using a DNP/anti-DNP pair. DNP is an antigen which specifically binds to anti-DNP antibody. Initial electro-fingerprints were obtained using a three-step process. First, a background spectrograph was obtained by using only a MOPS buffer solution so that the effects of the electrode/substrate combination (the chip) could be subtracted out from subsequent data collections.

Next, a 3 μl sample of 100 μg/ml anti-DNP was added to the chip surface and allowed to bind before a second impedance spectrograph was collected (e.g., step 50 of FIG. 7). The second spectrograph was background subtracted to remove the effects of the buffer and chip from the data. Then, a 3 μl sample of either 10 μg/ml DNP or a negative control, biotin, was added to the anti-DNP coated chip and allowed to bind for thirty seconds before another impedance spectrograph was collected. The background data was subtracted from the final spectrograph to remove the signal due to the buffer and chip from the data of the combined (DNP/anti-DNP or biotin/non-binding anti-DNP) proteins. The data from the intermediary stage (anti-DNP only) was subtracted from the final data to leave only the signal from the combined proteins.

The resultant data is shown in FIG. 8. Curve 61 represents a dissipation factor over a selected frequency range for bound DNP/anti-DNP, while curve 62 represents a dissipation factor over a selected frequency range for the biotin/non-binding anti-DNP control. Thus, a distinct electro-fingerprint is seen when a binding event occurs (curve 61) as opposed to when a binding event does not occur (curve 62) in the biosensor system.

The biosensor system was further tested to demonstrate the ability of the system to detect disease-relevant antibodies, i.e. disease markers. Fe1-d1 and Der-p1 are major proteins associated with allergic response in humans to cat and dustmite exposure, respectively. Separate IDEs were functionalized with these proteins utilizing ex-lipoic acid and EDC/NHS. Monoclonal antibodies against either Fe1-d1 or Der-p1 were added to the IDEs and allowed to react for 15 minutes (e.g., step 50 of FIG. 7). The IDEs were then washed with PBS-tween-20. Impedance changes following the addition of antibody and washing were measured and compared to a baseline measurement prior to adding the antibody. FIG. 9 shows the response of the anti-Fe1-d1 antibody in a system with electrodes functionalized with either Fe1-d1 (the binding antigen) or Der-p1 (used as a non-binding control antigen). A specific response was seen, with approximately a two-fold increase in signal achieved in the specific response compared to the non-specific response. In addition, the specific response was detectable at a concentration of 100 ng/ml.

Addition of functionalized nanoparticles to the biosensor system was also tested, similar to step 51 of FIG. 7 but without functionalizing the IDEs with first biomolecular probe. Two sets of nanoparticles, each composed of 10 nm gold nanoparticles, were functionalized through bio-conjugation of Protein A and goat anti-human IgG, respectively. As shown in FIG. 10, the two molecules, when used to functionalize the same amount and type of gold nanoparticles, produced distinctively different electro-fingerprints. Additionally, FIG. 10 shows that a combination of Protein A and anti-IgG itself produces an electro-fingerprint distinct from either Protein A or anti-IgG alone.

The difference in electro-fingerprints between biomolecules used to functionalize nanoparticles, as well as the difference in electro-fingerprints between biomolecules used to functionalize electrodes, can be used to determine what biomolecules are present in an unknown sample, and what binding has occurred. For instance, the frequencies at which peaks and valleys occur for a particular molecule can be stored as a specific fingerprint in a memory (e.g., profiler 44 of FIG. 6), which can then be used to check for positive or negative presence of the biomolecule in an unknown sample. While only phase curves are shown in FIG. 10, a person of ordinary skill in the art will recognize that other electrical parameters including impedance/conductance, capacitance, and dissipation factor can be measured with the system, as shown at step 53 of FIG. 7.

Further, the addition of functionalized nanoparticles to the biosensor system was shown to increase the sensitivity of the system over a system not using functionalized nanoparticles. The ability of a streptavidin (SA)-colloidal gold nanoparticle conjugate to yield an enhanced signal over the use of SA alone was tested. Electrodes were functionalized with a biotinylated 30-mer oligonucleotide, providing a biotinylated surface to which SA could bind. Two titration experiments were run on separate electrodes, the first to determine the binding characteristics of SA alone, and the second to test, under the same conditions, binding of the gold nanoparticle-SA conjugate.

The electrodes were initially incubated in a buffer solution of PBS-0.05% tween-20 to establish a stable background. After 30 minutes, SA at a concentration of 10 pM was added to the electrode and allowed to incubate for 15 minutes, during which impedance was monitored. These results are shown in FIG. 11A. The electrodes were next washed with a 1 ml flow-through of PBS-tween-20 and increasing ten-fold concentrations of SA were added to the electrode and allowed to incubate for 15 minutes per concentration. To obtain biosensor system sensitivity data for the gold nanoparticle-SA conjugate, the titration scheme was repeated in a similar fashion to the SA alone, starting with a gold nanoparticle-SA concentration of approximately 30 pM. These results are shown in FIG. 11B. The results of the titration experiment indicate that the gold nanoparticle-SA conjugate was able to generate a detectable impedance change at significantly lower concentrations as compared to SA alone.

FIG. 11A shows impedance data for increasing 10-fold concentrations of SA alone. The first spike indicates the addition of a 10 pM SA concentration to the electrode, at approximately 1843 seconds. Subsequent spikes indicate the addition of increasing 10-fold concentrations of SA. The arrow indicates the addition of 10 uM SA, corresponding to the first detectable change in impedance. Hence, the sensitivity of the system to SA alone was shown to be approximately 10 uM.

FIG. 11B shows impedance data for increasing 10-fold concentrations of gold nanoparticle-SA conjugate. After initial data collection, the first spike indicates the addition of a 30 pM concentration of gold nanoparticle-SA conjugate, at approximately 1595 seconds. Subsequent spikes indicate an increasing 10-fold concentration of gold nanoparticle-SA conjugate. The arrow indicates the addition of 30 nM gold nanoparticle-SA conjugate to the system, based upon SA concentrations of other gold nanoparticle-SA reagents. This corresponds to the first detectable change in impedance; hence, the sensitivity of the system to gold nanoparticle-SA conjugate is approximately 30 nM, a 300-fold increase in sensitivity over SA alone. While this increase in sensitivity was quite impressive, it should be noted that SA, possibly based upon the intrinsic electrical nature of the molecule and/or the size, does not produce a strong impedance change once bound to the electrode.

Nanoparticles used in the biosensor system can be functionalized to target specific disease markers. For instance, functionalized nanoparticles can be designed to measure c-reactive protein (CRP). CRP predicts future risk for cardiovascular diseases (CVD) including atherosclerosis, peripheral artery disease, myocardial infarction, and stroke in apparently healthy persons, independent of established risk factors. Originally, CRP was considered a simple indicator of inflammation, but emerging evidence suggests systemic inflammation may play a role in CVD by contributing to local plaque instability. Inflammation can activate the endothelium of arteries, which then expresses cellular adhesion molecules that recruit monocytes and low-density lipoproteins (LDL) into the coronary artery intima. LDL is then oxidized and taken up by macrophages that become activated and release cytokines and proteolytic enzymes. CRP binds to oxidized LDL through exposed oxidized phosphocholine (PC) and binds and clears apoptotic cells. Thus, there is a possible role for CRP as part of an innate response to oxidized PC-containing cells.

Because CRP binds to oxidized PC, PC-presenting metal nanoparticles that bind nCRP through multi-valent interactions can be designed. The lipid composition used can be varied to improve nanoparticle binding to nCRP. A series of lipid compositions can be tested that include a fraction of oxidized PC, the native binding element for nCRP. The size of the nanoparticle can be optimized to maximize sensitivity of the system. Affinity for nCRP can be established through the presentation of pentameric PC head groups exposed on the surface of the nanoparticles. Specificity for nCRP can arise from the chelate effect intrinsic to pentameric binding. In this manner, gold nanoparticles can be synthesized to mimic the high affinity biological substrates for CRP. While CRP has been used as a specific example, a person of ordinary skill in the art will recognize that any number of specific disease markers can be targeted using the biosensor system and methods described herein.

If the functionalized nanoparticles find the target molecules, they will bind to these molecules. The concept of impedance signature of the unbound functionalized molecules and these nanoparticles clumped together by the target molecules was tested using 10 nm gold nanoparticles functionalized with protein A and goat immunoglobin IgG. A representative frequency plot of phase theta is shown at FIG. 12, although frequency plots of impedance, capacitance, and dissipation factor may also be obtained. Three curves are shown: one with only nanoparticles functionalized with Protein A in the solution (Protein A), one with only nanoparticles functionalized with IgG in the solution (IgG), and one with a mixture of protein A- and IgG-functionalized nanoparticles in the solution (Protein A+IgG). In the latter curve, it is known that several nanoparticles functionalized with IgG will attach to the nanoparticles functionalized with Protein A, hence forming clumps. In each curve, the signal of the buffer solution was subtracted. The combination of nanoparticles functionalized with Protein A and nanoparticles functionalized with IgG produced a distinctively different signature from that seen with either nanoparticle alone.

Potential Future Applications

The biosensor system and a method for detecting and analyzing using the biosensor system has been described in relation to experiments which detected antibody-antigen reactions using defined, low complexity buffer systems. However, an ultimate goal of the biosensor system and method described herein is detection of these reactions (as well as other protein-protein interactions) in real-time, rapid one-step analysis of patient samples for disease applications. Further, a biosensor system may be configured with multiple IDEs and multiple pluralities of functionalized nanoparticles, for instance on a chip. Each IDE and its corresponding plurality of functionalized nanoparticles could be designed to probe for a different target biomolecule. In this manner, multiplexing could occur, and hence several disease or illness markers (such as antibodies) could be detected with one patient sample.

The specific example of CRP detection to determine risk of CVD has already been described. However, the biosensor system could also be used to determine risk for or presence of other diseases in patients and patient samples by looking for specific disease markers.

For instance, a test for Celiac sprue using the described biosensor system and method would be beneficial to the clinical diagnosis of the disease. Celiac sprue, or celiac disease, is an autoimmune disease that develops because of intolerance to ingested proteins (gluten) derived from wheat, rye, and barley. The disease is underdiagnosed, with an estimated incidence worldwide of 1 in 120-300 people. Currently, diagnosis of celiac disease requires sending patient samples to a clinical lab where multiple tests are performed. However, use of a diagnostic test based on the biosensor system described herein could allow for point-of-care, real-time, and multiplexing capabilities.

For example, a patient presenting symptoms of Celiac disease, such as severe intolerance to wheat gluten, may arrive at a doctor's office or hospital. A blood sample could be taken from the patient and serum could be collected from the blood sample. This serum could then be added to the biosensor system, which would include at least one functionalized interdigitated electrode and a plurality of nanoparticles that had been functionalized to detect antibodies for the disease. Rather than having to wait several days or weeks for results of the test, the patient could receive his results within a matter of minutes or hours, and a proper course of treatment for the patient could begin immediately. The same results could be seen with other disease markers conducive to use in the described biosensor system, including markers for cancer, heart disease, diabetes, and infectious disease.

A person skilled in the art will be able to practice the present invention in view of the description presented in this document, which is to be taken as a whole. Numerous details and examples have been set forth in order to provide a more thorough understanding of the invention. In other instances, well-known features have not been described in detail in order to not unnecessarily obscure the invention.

While the invention has been disclosed in its preferred form, the specific embodiments and examples thereof as disclosed and illustrated herein are not to be considered in a limiting sense. It should be readily apparent to those skilled in the art in view of the present description that the invention can be modified in numerous ways. The inventor regards the subject matter of the invention to include all combinations and sub-combinations of the various elements, features, functions and/or properties disclosed herein.

1. A method of detecting and analyzing biomolecules in a sample analyte, the method comprising:
functionalizing a plurality of metallic nanoparticles;
providing a plurality of electrodes, each electrode having an exposed surface;
applying the plurality of functionalized metallic nanoparticles and the sample analyte to the plurality of electrodes; and
using impedance spectroscopy to detect a sample signal profile for a group of sample electrical parameters across a selected frequency range.

2. The method of claim 1, wherein the plurality of electrodes are functionalized by coating the exposed surfaces with a plurality of first biomolecular probes.

3. The method of claim 2, wherein the first biomolecular probe includes at least one of a group of proteins consisting of an antigen, an antibody, a secondary antibody, an isotype, an enzyme, a nucleic acid, a cytokine, and a peptide.

4. The method of claim 2, wherein the first biomolecular probe is specific for a disease marker.

5. The method of claim 2, wherein functionalizing the plurality of metallic electrodes includes first coating the exposed surface with a layer comprising a polymer or a silicon oxide.

6. The method of claim 1, wherein the plurality of metallic nanoparticles is functionalized by coating an outer surface of the metallic nanoparticles with a plurality of second biomolecular probes.

7. The method of claim 6, wherein the second biomolecular probe includes at least one of a group of proteins consisting of an antigen, an antibody, a secondary antibody, an isotype, an enzyme, a nucleic acid, a cytokine, and a peptide.

8. The method of claim 6, wherein the second biomolecular probe is specific for a disease marker.

9. The method of claim 6, wherein functionalizing the plurality of metallic nanoparticles includes first coating the outer surface with a layer comprising a polymer, a silicon oxide, or a surfactant.

10. The method of claim 1, wherein the plurality of electrodes is formed in an interdigitated relationship, the electrodes having multiple fingers with opposed side walls at a spacing of about an order of magnitude greater than a dimension of the metallic nanoparticles.

11. The method of claim 1, wherein each electrode has an exposed surface formed of a metal such as chromium, gold, iron, platinum, or palladium.

12. The method of claim 1, wherein the plurality of functionalized metallic nanoparticles is applied to the plurality of electrodes separately from the sample analyte.

13. The method of claim 1, wherein the plurality of functionalized metallic nanoparticles are combined with the sample analyte to form a mixture that is then applied to the plurality of electrodes.

14. The method of claim 1, wherein the plurality of metallic nanoparticles comprise gold nanoparticles, silver nanoparticles, iron nanoparticles, iron oxide nanoparticles, platinum nanoparticles, or palladium nanoparticles.

15. The method of claim 1, wherein the group of sample electrical parameters comprises one or more of an impedance, a capacitance, a dissipation factor, and a phase.

16. The method of claim 1, wherein the selected frequency range includes 25 Hz to 50 kHz.

17. The method of claim 1, further comprising comparing the sample signal profile with a reference sample signal profile across the selected frequency range.

18. A biosensor system for detecting or identifying biomolecules in a sample analyte, the biosensor system comprising:
a substrate;
an electrode formed on the substrate, the electrode including one or more pairs of opposed fingers, each finger having an exposed upper surface and exposed side walls;
a stimulator electrically coupled to the electrode and structured to provide a plurality of input frequencies over a selected frequency range;
a detector operative to detect a signal of the sample analyte over the selected frequency range and generate a sample signal profile for a group of sample electrical parameters;
means for comparing the sample signal profile with a reference signal profile across the selected frequency range; and
a plurality of functionalized nanoparticles.

19. The biosensor system of claim 18, further comprising a sample channel structured to contain the functionalized electrode such that a flow of the sample analyte can be introduced to the system.

20. The biosensor system of claim 18, wherein the electrode is functionalized with a plurality of first biomolecular probes selected from a group of proteins consisting of an antigen, an antibody, a secondary antibody, an isotype, an enzyme, a nucleic acid, a cytokine, and a peptide.

21. The biosensor system of claim 18, wherein the exposed upper surface and the exposed side walls are formed of a metal such as chromium, gold, iron, platinum, or palladium.

22. The biosensor system of claim 18, wherein the fingers of the electrode are spaced with a gap of a size greater than both a length and a width of the functionalized nanoparticles.

23. The biosensor system of claim 18, wherein the stimulator is operative to provide a selected frequency range which includes 25 Hz to 50 kHz.

24. The biosensor system of claim 18, wherein the detector is operative to detect a group of sample electrical parameters which includes one or more of an impedance, a capacitance, a dissipation factor, and a phase.

25. The biosensor system of claim 18, wherein the functionalized nanoparticles are functionalized with a plurality of second biomolecular probes selected from a group of proteins consisting of an antigen, an antibody, a secondary antibody, an isotype, an enzyme, a nucleic acid, a cytokine, and a peptide.

26. The biosensor system of claim 18, wherein the functionalized nanoparticles comprise at least one of a metal such as gold, silver, iron, iron oxide, platinum, or palladium.

27. The biosensor system of claim 18, wherein the functionalized nanoparticles further comprise functionalized nanowires or functionalized nanotubes.

28. A method of detecting and analyzing biomolecules in a reference sample analyte, the method comprising:
functionalizing a plurality of metallic nanoparticles;
providing a plurality of electrodes, each electrode having an exposed surface;
applying the plurality of functionalized metallic nanoparticles and the reference sample analyte to the plurality of electrodes; and
using impedance spectroscopy to detect a reference sample signal profile for a group of sample electrical parameters across a selected frequency range.

29. The method of claim 28, wherein the reference sample signal profile is stored in a database for future comparison to a detected sample signal profile of a sample analyte.

30. The method of claim 28, further comprising subtracting a signal of a buffer solution from a reference sample signal to obtain the reference sample signal profile.

31. The method of claim 30, wherein the plurality of electrodes are functionalized by coating the exposed surfaces with a plurality of first biomolecular probes, the first biomolecular probes including at least one of a group of proteins consisting of an antigen, an antibody, a secondary antibody, an isotype, an enzyme, a nucleic acid, a cytokine, and a peptide.

32. The method of claim 30, wherein plurality of metallic nanoparticles is functionalized by coating an outer surface of the metallic nanoparticles with a plurality of second biomolecular probes, the second biomolecular probes including at least one of a group of proteins consisting of an antigen, an antibody, a secondary antibody, an isotype, an enzyme, a nucleic acid, a cytokine, and a peptide.
